SEQUENCE LISTING (2 sequences)

SEQ ID NO: 1 - DNA, 1508 bp, Lactobacillus kefiri
  modified_base (1477)..(1477): a, c, t, g, unknown or other
  modified_base (1487)..(1493): a, c, t, g, unknown or other
  modified_base (1495)..(1501): a, c, t, g, unknown or other
cctagcgtgcccacaggtttgacacggcgcaaaaatctcaccaggtggccgcacagagtgtgatcccggctcactaaatctctccacagggggcccacacagagtgttccccccgctctcaatatctcaaaaaagggcccacacacagtgtttttcccgctcacaaactaaaaccacagggtctccacatatattttttcttccgctatcactttaagatggacccgcggcctattagcttgttggtaaggtaatggcctaccaaggcaatgatacgtagccgacctgagagggtaatcggccacattgggactgagacacggcccaaactcctacgggaggcagcagtagggaatcttccacaatggacgaaagtctgatggagcaacgccgcgtgagtgatgaagggtttcggctcgtaaaactctgttgttggagaagaacaggtgtcagagtaactgttgacatcttgacggtatccaaccagaaagccacggctaactacgtgccagcagccgcggtaatacgtaggtggcaagcgttgtccggatttattgggcgtaaagcgagcgcaggcggtttcttaggtctgatgtgaaagccttcggcttaaccggagaagtgcatcggaaaccaggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgatgctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattgttagttgccagcattcagttgggcactctagcaagactgccggtgacaaaccggaggaaggtggggatgacgtcaaatcatcatgccccttatgacctgggctacacacgtgctacaatggacggtacaacgagtcgcgaaaccgcgaggtcaagctaatctcttaaagccgttctcagttcggattgcaggctgcaactcgcctgcatgaagttggaatcgctagtaatcgtggatcagcatgccacggtgaatacgttcccgggccttgtacacaccgcccgtcacaccatgagagtttgtaacacccaaagccggtgaggtaaccttcggggaccagccgtctaaggtgggacaatgattanggtgagtcannnnnnngnnnnnnncaaaata

SEQ ID NO: 2 - DNA, 19 bp, Artificial Sequence
  Description of Artificial Sequence Synthetic primer
gagtttgatcctggctcag

(19) United States
(12) Patent Application Publication
(10) Pub. No.: US 2017/0252392 A1
(43) Pub. Date: Sep. 7, 2017

(54) COMPOSITION FOR IMPROVEMENT, TREATMENT OR PREVENTION OF CONSTIPATION COMPRISING CASSIA SEED LACTIC ACID BACTERIA FERMENTED PRODUCT AS EFFECTIVE INGREDIENT, AND METHOD FOR PREPARING SAME

(71) Applicant: HAENAM NATURAL FARMING ASSOCIATION COOPERATION, Jeollanam-do (KR)

(72) Inventors: Sang Duk HWANG, Jeollanam-do (KR); Gum Sul YONG, Jeollanam-do (KR); Ju Hyun YONG, Jeollanam-do (KR); Ju Sun YONG, Jeollanam-do (KR); Yong Su SONG, Gwangju (KR)

(73) Assignee: HAENAM NATURAL FARMING ASSOCIATION COOPERATION, Jeollanam-do (KR)

(21) Appl. No.: 15/318,246

(22) PCT Filed: Oct. 27, 2015
(86) PCT No.: PCT/KR2015/011359

§ 371 Date: Dec. 12, 2016

(30) Foreign Application Priority Data
Nov. 4, 2014 (KR) 10-2014-0151888

(51) Int. Cl. A61K 36/482; A23L 33/00; A23L 33/105; C12N 1/20; C12P 1/04

(52) U.S. Cl. CPC A61K 36/482; C12N 1/20; C12P 1/04; A23L 33/105; A23L 33/40; A61K 2236/19; A61K 2236/33; A23V 2002/00; A23Y 2220/47

(57) ABSTRACT

The present invention relates to a lactic acid fermented product of cassia seeds. The fermented product of the present invention uses cassia seeds, which have long been used and have proven their safety without causing side effects. Due to this advantage, the lactic acid fermented product of the present invention is effective in increasing the number, water content, and/or weight of feces. The effects of the lactic acid fermented product according to the present invention are comparable to those of sennoside, which is a therapeutic agent for stimulant constipation and whose use in health functional foods is forbidden because of its side effects, and Dulcolax-S, which is currently commercially available and includes bisacodyl and sodium docusate as active ingredients. Therefore, the lactic acid fermented product of the present invention can be utilized in pharmaceutical compositions for treating or preventing constipation or health functional foods for ameliorating or preventing constipation. In addition, the present invention relates to Lactobacillus kefiri strain MJ90. The strain is advantageous for the preparation of the lactic acid fermented product with enhanced activity in the treatment, amelioration or prevention of constipation.

### TECHNICAL FIELD

The present invention relates to a health functional food for ameliorating or preventing constipation and a pharmaceutical composition for treating or preventing constipation which include, as an as active ingredient, a lactic acid fermented product of cassia seeds to increase the water content and weight of feces. The present invention also relates to a method for preparing a lactic acid fermented product of cassia seeds and a strain used in the method.

### BACKGROUND ART

Constipation is medically defined as having a bowel movement three times or fewer per week or passing a small amount of feces (≦30 g) per day. In a normal state, feces are excreted within 24 hours after food ingestion. People with constipation suffer from infrequent stools only once several days or defecate only a small quantity of feces a day. In conclusion, ingesta stay longer in the stomachs and intestines of people who have constipation.

Many causes are responsible for constipation, such as poor colonic peristalsis due to skipping breakfast, a lack of dietary fiber due to westernization of dietary habits, low level of basic physical fitness and weak abdominal muscles due to a lack of exercise, dieting, and drug addiction due to an overdose.

From ancient times, constipation has been considered as the basic cause of all diseases. Constipation is characterized by loss of appetite and continuous abdominal distension. In people with constipation, toxins present in unexcreted feces are absorbed into the blood through the intestines, increasing the risk of early skin aging and undesirable symptoms, such as headache, acne, and rash. Severe constipation is a cause of hemorrhoids, such as anal fissure tear and hemorrhoidal protrusion, upon defecation and even toxins in feces are known to be involved in the initiation of colorectal cancer. When constipation persists, cholesterols and fats remain in the body without being excreted, causing arteriosclerosis or cholelithiasis. Furthermore, constipation may induce hypertension and cardiac hypertrophy by which heart disease may be exacerbated. Constipation is a cause of diverse and serious secondary diseases, such as stroke, immunodeficiency, visual disturbance, and mental disease (depression). For these reasons, constipation is considered a serious disease that needs to be actively prevented and treated.

Various kinds of medicines and health functional foods are currently commercially available for the treatment or amelioration of constipation. Most of them use stimulant medications containing anthraquinone derivatives, such as senna and rhubarb. These stimulant medications cause side effects, such as stomachache and diarrhea, and problems when taken during pregnancy or continuously taken. Most of the health functional foods fail to scientifically prove their effects. The fundamental problems associated with the amelioration of constipation remain unsolved, leading to poor product reliability.

Cassia is an annual herbaceous belonging to the family Leguminosae and is known to include two species: Cassia obtusifolia L. and Cassia tora L. Cassia seed is also called “chogyeolmyeong” or “gingangnamcha” in Korean. Cassia obtusifolia L. indigenous to Central America is cultivated in Japan and elsewhere. Cassia tora L. indigenous to Tropical Asia is cultivated in Korea, China, and elsewhere.

Cassia fruit has an irregular hexagonal columnar shape with one sharp side and is light or dark brown in color, 3-6 mm in length, and 2-3.5 mm in diameter. Cassia seed shell is hard and glossy, has glossy narrow engraved stripes at both sides, and has a distinct smell. C. obtusifolia has large-grain seeds and C. tora has small-grain seeds.

Cassia seeds are known to clear away heat and make the eyes clean in the Oriental medicine field. Due to these effects, cassia seeds are used to treat acute conjunctivitis, ocular hemorrhage, an uncomfortable feeling or pain in the eye, corneal opacity in the eyes of the elderly when tearing, i.e. chronic progressive disease, early amblyopia caused by optic nerve and retinal atrophy, obesity, hypertension, headache caused by metal stress, tinnitus, and eye tumors. It is known that cassia seeds treat hyperpiesia and dilate the coronary artery to reduce the resistance of blood flow, contributing to blood pressure reduction. Cassia seeds are also known to treat hemiplegia caused by stroke, mild diarrhea, and chronic diseases. Another function of cassia seeds is to reduce the level of cholesterol. Due to this function, cassia seeds are used to treat cardiovascular diseases. Specifically, when the total cholesterol is beyond the normal level, cassia seeds are used to treat angina caused by coronary sclerosis. Cassia seeds are also known to treat senile constipation, senile hyperpiesia, mouth dryness caused by a lack of bodily humor, abdominal distension caused by constipation, and habitual constipation.

Korean Patent Publication No. 1999-0084271 describes a tea bag prepared by mixing cassia seeds or leaves with other herbal medicines, simply pulverizing the mixture into a powder, and roasting the powder, and explains that the tea bag is effective in ameliorating constipation. Further, Korean Patent Publication No. 2003-0062493 describes a leached tea prepared by extracting cassia seeds, concentrating the extract, spray drying the concentrate to prepare a powder, and mixing the powder with a powder of a Cascara sagrada extract, and explains that the leach tea is highly palatable and effective in ameliorating constipation symptoms. According to these patent publications, cassia seeds are simply pulverized and roasted, or extracted, concentrated, and dried, or mixed with other herbal medicines which are effective in ameliorating constipation.

Under these circumstances, the present inventors have found that a lactic acid fermented product of cassia seeds, which have been proven to be safe, increases the number, water content or weight of feces and is thus very effective in ameliorating or treating constipation, compared to cassia seed extracts. The present invention has been achieved based on this finding.

### DETAILED DESCRIPTION OF THE INVENTION

### Problems to be Solved by the Invention

It is one object of the present invention to provide a health functional food for ameliorating or preventing constipation that uses cassia seeds as herbal medicines, which have long been used and have proven their safety without causing side effects, achieving markedly enhanced water content and weight of feces.

It is a further object of the present invention to provide a pharmaceutical composition for treating or preventing constipation that uses cassia seeds to achieve markedly enhanced water content and weight of feces.

It is another object of the present invention to provide a method for preparing a lactic acid fermented product of cassia seeds that is effective in ameliorating or preventing constipation.

It is still another object of the present invention to provide a novel strain that can enhance the activity of cassia seeds in the amelioration or prevention of constipation.

### Means for Solving the Problems

The present invention provides a health functional food for ameliorating or preventing constipation including, as an active ingredient, a lactic acid fermented product of cassia seeds.

The cassia seeds may be extracted with water, a C₁-C₄ alcohol or a mixed solvent thereof before fermentation.

The health functional food may be in the form of a capsule, tablet, powder, granule, liquid, pill, flake, paste, syrup, gel, jelly or bar.

The present invention also provides a pharmaceutical composition for treating or preventing constipation including, as an active ingredient, a lactic acid fermented product of cassia seeds.

The cassia seeds may be extracted with water, a C₁-C₄ alcohol or a mixed solvent thereof before fermentation.

The present invention also provides a method for preparing a lactic acid fermented product of cassia seeds, including: preparing a cassia seed extract; and inoculating lactic acid bacteria into the cassia seed extract, followed by fermentation wherein the lactic acid fermented product increases the number, water content, and/or weight of feces.

The lactic acid bacteria may be selected from the group consisting of lactic acid bacteria belonging to the genera Lactobacillus, Bifidobacterium, Leuconostoc, Pediococcus, and Enterococcus.

The lactic acid bacterial strain may be Lactobacillus kefiri MJ90 [Deposit number: KCCM 11575P].

The cassia seeds or their powder is extracted with water, a C₁-C₄ alcohol or a mixed solvent thereof; and the cassia seed extract is inoculated with 1×10³ to 1×10⁶ CFU/ml of Lactobacillus kefiri MJ90 [Deposit number: KCCM115751P] and fermented at 30 to 45° C. for 1 to 7 days.

The extraction may be performed by mixing 1 part by weight of the cassia seed powder with 10 to 50 parts by weight of water and allowing the mixture to stand or stirring the mixture for 0.5 to 24 hours.

The extraction may be performed by extracting 1 part by weight of the cassia seeds or their powder with 20 to 80% by weight of an aqueous ethanolic solution for 0.5 to 24 hours and drying the extract to remove the ethanol.

The cassia seed extract may be fermented in the presence of one or more nutrients for the lactic acid bacteria.

The present invention also provides Lactobacillus kefiri MJ90 (deposit number: KCCM11575P) that ferments cassia seeds to enhance the activity of the cassia seeds in the amelioration or prevention of constipation.

The present invention also provides a method for treating or preventing constipation, including administering to a patient in need of such treatment or prevention an effective amount of a lactic acid fermented product of cassia seeds.

### Effects of the Invention

The lactic acid fermented product of the present invention uses cassia seeds, which have long been used and have proven their safety without causing side effects. Due to this advantage, the lactic acid fermented product of the present invention is effective in increasing the number, water content, and/or weight of feces. The effects of the lactic acid fermented product are comparable to those of sennoside, which is a therapeutic agent for stimulant constipation and whose use in health functional foods is forbidden because of its side effects, and Dulcolax-S, which is currently commercially available and includes bisacodyl and sodium docusate as active ingredients. Therefore, the lactic acid fermented product of the present invention can be utilized in pharmaceutical compositions for treating or preventing constipation or health functional foods for ameliorating or preventing constipation. In addition, Lactobacillus kefiri strain MJ90 of the present invention is advantageous for the preparation of the lactic acid fermented product with enhanced activity in the treatment, amelioration or prevention of constipation.

### BRIEF DESCRIPTION OF THE DRAWINGS

FIG. 1 shows the progress of lactic acid fermentation of cassia seed extracts of Preparative Examples 1 (1a) and 2 (1b) and a dilution of Preparative Example 3 (1c) with different microorganisms through changes in pH measured at 36 and 72 hours after fermentation in Experimental Example 1.

FIG. 2 shows the numbers of feces from experimental groups, which were measured in C of Experimental Example 2.

FIG. 3 shows the weights of feces from experimental groups, which were measured in C of Experimental Example 2.

FIG. 4 shows the numbers of feces from experimental groups, which were measured in C of Experimental Example 3.

FIG. 5 shows the weights of feces from experimental groups, which were measured in C of Experimental Example 3.

FIG. 6 shows images of washed colons excised from sacrificed experimental animals in D of Experimental Example 3 (the points under the images represent the colonic transits of carmine dye).

FIG. 7 shows the colonic transits of carmine dye measured from the images of FIG. 6.

FIG. 8a shows an HPLC-IT/TOF MS pattern of an ethanolic cassia seed extract prepared in Preparative Example 2, which was recorded in Experimental Example 4, and FIG. 8b shows an HPLC-IT/TOF MS pattern of a lactic acid fermented product of cassia seeds prepared in Preparative Example 4, which was recorded in Experimental Example 4.

FIG. 9 shows the lineage of Lactobacillus kefiri strain MJ90, which was isolated and identified in Experimental Example 9.

### BEST MODE FOR CARRYING OUT THE INVENTION

The present invention is directed to a health functional food for ameliorating or preventing constipation including, as an active ingredient, a lactic acid fermented product of cassia seeds. The present invention is also directed to a pharmaceutical composition for treating or preventing constipation including, as an active ingredient, a lactic acid fermented product of cassia seeds. The invention is also directed to a method for preparing a lactic acid fermented product of cassia seeds, including: preparing a cassia seed extract; and inoculating lactic acid bacteria into the cassia seed extract, followed by fermentation wherein the lactic acid fermented product increases the number, water content, and/or weight of feces.

The cassia is Cassia obtusifolia L. or Cassia tora L., preferably Cassia tora L. that is cultivated in Korea.

The extract is intended to include not only crude extracts obtained by extracting the raw materials with solvents, but also products obtained by processing the crude extracts. For example, the cassia seed extract may be prepared into a powder by additional processes, such as distillation under reduced pressure and freeze drying or spray drying. The extract is also intended to include fractions obtained by fractionation of the crude extracts.

The lactic acid bacteria may be selected from the group consisting of lactic acid bacteria belonging to the genera Lactobacillus, Bifidobacterium, Leuconostoc, Pediococcus, and Enterococcus. For example, the lactic acid bacterial strain may be Lactobacillus paracasei, Lactobacillus kefiri, Lactobacillus acidophilus, Bifidobacterium longum, Bifidobacterium brevis, Leuconostoc mesenteroides, Pediococcus pentosaceus or Enterococcus faecalis. The lactic acid bacterial strain is preferably a Lactobacillus sp., more preferably Lactobacillus kefiri or Lactobacillus acidophilus, most preferably Lactobacillus kefiri MJ90, which was newly isolated and identified by the present inventors.

Specifically, cassia seeds or their powder is extracted with water, a C₁-C₄ alcohol or a mixed solvent thereof; and the cassia seed extract is inoculated with 1×10³ to 1×10⁶ CFU/ml of lactic acid bacteria and fermented at 30 to 45° C. for 1 to 7 days. That is, the lactic acid fermented product can be obtained by inoculating lactic acid bacteria into a cassia seed extract and fermenting the inoculated cassia seed extract.

The extraction solvent may be water. In this case, 1 part by weight of the cassia seed powder is diluted with 10 to 50 parts by weight of cold water or water at room temperature to extract ingredients from the cassia seeds. The extraction may be performed separately from fermentation. Alternatively, ingredients may be dissolved from the cassia seeds inoculated with lactic acid bacteria during fermentation. Accordingly, in a broad sense, the term “cassia seed extract” includes a solution of the cassia seed powder diluted in water. Aqueous dilutions of cassia seeds are somewhat less effective in ameliorating, treating or preventing constipation than products obtained by fermentation of hot water or ethanolic extracts of cassia seeds with lactic acid bacteria but are significantly effective in ameliorating, treating or preventing constipation compared to non-fermented cassia seed extracts, which can be seen from the Experimental Examples section that follows.

For better extraction efficiency, the extraction solvent may be hot water. In this case, 1 part by weight of cassia seeds or their powder may be mixed with 10 to 50 parts by weight of water at 80 to 105□, preferably 90 to 100□, and the mixture is allowed to stand or stirred for 0.5 to 24 hours, preferably 1 to 6 hours, to obtain a cassia seed extract.

The solvent may be a C₁-C₄ alcohol or an aqueous solution thereof. In this case, 1 part by weight of cassia seeds or their powder may be extracted with 5 to 30 parts by weight of the solvent for 0.5 to 24 hours. For example, cassia seeds or their powder may be extracted with an aqueous solution of an alcohol, such as ethanol, methanol or isopropanol. The aqueous alcoholic solution is preferably used in an amount of 20 to 80% by weight, more preferably 50 to 70% by weight. When the alcoholic extract is used, the alcohol is removed from the alcoholic extract by evaporation before inoculation with lactic acid bacteria to obtain a concentrate with a lower alcohol content. Alternatively, the alcoholic extract may be concentrated, dried, and dissolved in water before use.

Before inoculation of lactic acid bacteria into the cassia seed extract, it is necessary to enhance the growth of the lactic acid bacteria. To this end, the cassia seed extract may be further mixed with one or more nutrient sources for the lactic acid bacteria. For example, the nutrient sources may be selected from protein sources, carbohydrate sources, vitamins, and minerals. In addition, a starter may be proliferated or activated in the presence of a mixture of the nutrient sources and lactic acid bacteria before inoculation of the lactic acid bacteria into the cassia seed extract. The nutrient sources may be obtained from commercially available media. Only necessary nutrients may be individually added to the cassia seed extract.

The cassia seed extract or the mixture of the cassia seed extract and the nutrient sources may be sterilized by heating before inoculation with lactic acid bacteria.

The solid content of the cassia seed extract is not particularly limited and may be from 1 to 15% by weight, for example, when no additional concentration is performed after extraction. The cassia seed extract may be directly used. Alternatively, the cassia seed extract may be concentrated before use. The cassia seed extract may also be concentrated or dried and diluted before use.

Another lactic acid bacterial strain may be added during or after fermentation with Lactobacillus kefiri strain MJ90. The addition of the auxiliary lactic acid bacterial strain is effective for intestinal regulation for the amelioration or prevention of constipation,

The lactic acid bacteria may be selected from the group consisting of lactic acid bacteria belonging to the genera Lactobacillus, Bifidobacterium, Leuconostoc, Pediococcus, and Enterococcus.

The fermentation conditions are the same as general lactic acid fermentation conditions and are thus not particularly limited. For example, the fermentation may be performed at 25 to 40□ for 24 to 96 hours.

The lactic acid fermented product of cassia seeds may include the lactic acid bacteria used. The lactic acid bacteria may be removed from the lactic acid fermented product. For example, the lactic acid bacteria may be killed by sterilization of the lactic acid fermented product. The lactic acid fermented product may be a filtrate obtained by filtration or a supernatant obtained by centrifugation.

The desired effect of the fermented product on the amelioration, treatment or prevention of constipation is believed to be because the ingredients of the cassia seed extract are biologically converted to ingredients suitable for in vivo use or novel active ingredients effective in ameliorating, treating or preventing constipation are produced during proliferation of the lactic acid bacteria. This effect is more profound than the effect obtained when lactic acid bacteria are simply added. The lactic acid fermented product of cassia seeds may be in the form of a liquid that can be used directly. Alternatively, the lactic acid fermented product may be dried and pulverized into a powder before use.

The lactic acid fermented product of cassia seeds can be used as an active ingredient of a health functional food for ameliorating or preventing constipation or a pharmaceutical composition for treating or preventing constipation.

As used herein, the term “including as an active ingredient” means that the lactic acid fermented product of cassia seeds is present in an amount sufficient to achieve the desired efficacy or activity in the amelioration, treatment or prevention of constipation.

The health functional food may be prepared by formulating the lactic acid fermented product of cassia seeds into a capsule, tablet, powder, granule, liquid, pill, flake, paste, syrup, gel, jelly or bar. The health functional food may be in the form of a general food, such as a beverage, tea, spice, gum or snack. In this case, the lactic acid fermented product of cassia seeds is added to the food material. The intake of the health functional food brings about the desired healthy effect. The health functional food causes no side effects even when taken for a long time, unlike general medicines.

The health functional food is very useful because its routine intake is possible. The content of the lactic acid fermented product of cassia seeds in the health functional food varies depending on the kind of the health functional food and is thus not uniformly determined. The content of the lactic acid fermented product of cassia seeds is not particularly limited so long as the lactic acid fermented product does not change the original taste of the food. The lactic acid fermented product of cassia seeds is typically added in an amount of 0.01 to 50% by weight, preferably 0.1 to 20% by weight, based on the weight of the desired food. In the case of the health functional food in the form of a capsule, tablet, powder, granule, liquid, pill, flake, paste, syrup, gel, jelly or bar, the lactic acid fermented product is typically added in an amount of 0.1 to 100% by weight, preferably 0.5 to 80% by weight. The daily dose of the lactic acid fermented product of cassia seeds is from 0.001 to 10 g/kg, preferably from 100 to 1000 mg/kg, more preferably from 200 to 500 mg/kg.

In addition to the active ingredient, the health functional food may optionally further include one or more ingredients that are usually added for food production. For example, the optional ingredients may be selected from proteins, carbohydrates, fats, nutrients, seasoning agents, and flavoring agents. Examples of the carbohydrates include: saccharides, such as monosaccharides (e.g., glucose and fructose), disaccharides (e.g., maltose, sucrose, and oligosaccharides), and polysaccharides (e.g., dextrin and cyclodextrin); and sugar alcohols, such as xylitol, sorbitol, and erythritol. Natural flavoring agents, such as thaumartin and stevia extracts (e.g., rebaudioside A and glycyrrhizin) and synthetic flavoring agents (e.g., saccharin and aspartame) may be used. For example, the health functional food of the present invention may be prepared into a drink or beverage. In this case, the health functional food of the present invention may further include citric acid, high fructose corn syrup, sugar, glucose, acetic acid, malic acid, fruit juice, and a plant extract, in addition to the active ingredient.

The pharmaceutical composition for treating or preventing constipation includes, for example, 0.001 mg/kg or more, preferably 0.1 mg/kg or more, more preferably 10 mg/kg or more, even more preferably 100 mg/kg or more, still even more preferably 250 mg/kg or more of the lactic acid fermented product of cassia seeds as an active ingredient. For example, the pharmaceutical composition may include 200 to 500 mg/kg of the lactic acid fermented product of cassia seeds. The lactic acid fermented product of naturally occurring cassia seeds causes no side effects in humans even when administered in an excessive amount. Therefore, the upper limit of the content of the lactic acid fermented product in the composition of the present invention can be appropriately determined by those skilled in the art.

The pharmaceutical composition may further include one or more pharmaceutically suitable and physiologically acceptable adjuvants, in addition to the active ingredient. Examples of the adjuvants include excipients, disintegrants, sweeteners, binders, encapsulating agents, swelling agents, lubricants, glidants, and flavoring agents.

The pharmaceutical composition may be formulated into a suitable dosage form for administration. In this case, the pharmaceutical composition may include at least one pharmaceutically acceptable carrier, in addition to the active ingredient.

For example, the pharmaceutical composition may be formulated into a granule, powder, tablet, coated tablet, capsule, suppository, solution, syrup, juice, suspension, emulsion, drop or injectable solution. For example, the pharmaceutical composition may be formulated into a tablet or capsule. For this formulation, the active ingredient may be combined with an oral, nontoxic, pharmaceutically acceptable inert carrier, such as ethanol, glycerol or water. If desired or necessary, suitable binders, lubricants, disintegrants, and coloring agents may be included as a mixture. Suitable binders include, but are not limited to, natural sugars, such as starch, gelatin, glucose, and beta-lactose, natural and synthetic gums, such as corn sweeteners, acacia, tragacanth, and sodium oleate, sodium stearate, magnesium stearate, sodium benzoate, sodium acetate, and sodium chloride. Suitable disintegrants include, but are not limited to, starch, methylcellulose, agar, bentonite, and xanthan gum.

The composition may be formulated into a solution. In this case, the composition may further include a pharmaceutically acceptable carrier. Suitable pharmaceutically acceptable carriers are sterile biocompatible carriers and examples thereof include saline, sterilized water, Ringer's solution, buffered saline, albumin injectable solution, dextrose solution, maltodextrin solution, glycerol, and ethanol, which may be used alone or as a mixture of two or more thereof. If necessary, the composition may further include other general additives, such as antioxidants, buffers, and bacteriostatic agents. Diluents, dispersants, surfactants, binders, and lubricants may be further added to prepare injectable formulations (such as aqueous solutions, suspensions, and emulsions), pills, capsules, granules, and tablets.

The composition can be formulated according to the type of diseases or the kind of ingredients in accordance with any suitable method known in the art, preferably, any of the methods disclosed in Remington's Pharmaceutical Science, Mack Publishing Company, Easton Pa.

The pharmaceutical composition may be administered orally or parenterally. The pharmaceutical composition is administered parenterally, for example, intravenously, subcutaneously, intramuscularly, intraperitoneally or transdermally, but is preferably administered orally.

The dose of the pharmaceutical composition may vary depending on various factors, such as the formulation method, the mode of administration, the age, body weight, sex, condition, and diet of patient, the time and route of administration, the rate of excretion, and the responsiveness of the condition. The effective dose of the pharmaceutical composition for the desired treatment or prevention can be readily determined by the judgement of skilled physicians. According to a preferred embodiment, the daily dose of the pharmaceutical composition is from 0.001 to 10 g/kg, preferably from 100 to 1000 mg/kg, more preferably from 200 to 500 mg/kg.

The pharmaceutical composition may be formulated with a pharmaceutically acceptable carrier and/or excipient according to suitable methods which can be easily carried out by one of ordinary skill in the art to which the present invention belongs. The formulation may be provided in a unit dosage form or a multi-dose container. The formulation may also be in the form of a solution, suspension or emulsion in an oil or aqueous medium, an elixir, a powder, a granule, a tablet or a capsule and may further include a dispersant or a stabilizer.

### MODE FOR CARRYING OUT THE INVENTION

The present invention will be described in more detail with reference to the following examples. These examples are provided to assist in understanding the invention and the scope of the invention is not limited thereto.

### Preparative Example 1: Preparation of Hot Water Extract from Cassia Seeds

The seeds of Cassia tora L. purchased from the Jeonnam Herb Medicine Agricultural Cooperative (Hwasun-gun), Korea, were selected carefully, washed with water, and uniformly pulverized with a pulverizer to obtain a powder having an average particle size of 300 μm. The cassia seed powder was kept refrigerated before use.

The cassia seed powder was extracted twice with a 10-fold amount of distilled water in an extraction water bath at 100° C. for 3 h. The extract was allowed to cool, filtered, concentrated using an evaporator, and freeze-dried to obtain an extract powder. The yield was 2.2%.

### Preparative Example 2: Preparation of Aqueous Ethanolic Extract from Cassia Seeds

The cassia seed powder obtained in Preparative Example 1 was extracted with a 4-fold amount of an aqueous ethanolic solution at room temperature for 3 days, filtered, concentrated using an evaporator, and freeze-dried to obtain an aqueous ethanolic extract powder. The yield was 9.8%.

### Preparative Example 3: Preparation of Aqueous Dilution of Cassia Seeds

Water was mixed with the cassia seed powder obtained in Preparative Example 1 to prepare a 7 wt % dilution of the cassia seeds.

### Preparative Example 4: Preparation of Fermented Products of Cassia Seeds

Each of the extract powders obtained in Preparative Examples 1 and 2 was diluted with distilled water to 5 wt %. Each of the extract dilution and the aqueous dilution of Preparative Example 3 was sterilized at 121□ and 1.5 atm for 15 min. Then, the lactic acid bacterial strains shown in Table 1 were activated by stationary culture in MRS broth and GAM broth at 37□ for 24 h. 5 wt % (1×10⁷ CFU/ml) of the culture broth was inoculated into the sterilized dilution, followed by culturing with stirring at 150 rpm at 37□ for 72 h to obtain a culture solution.

5 wt % (1×10⁷ CFU/ml) of a culture broth of Saccharomyces cerevisiae, a commercial baking yeast, which had previously been activated, were inoculated into the cassia seed extract (solid content: 5%) of Preparative Example 2 and cultured with stirring at 150 rpm at 30□ for 72 h to obtain a culture solution.

The culture solutions were centrifuged at 6,000 rpm at 4° C. for 15 min and the resulting bacteria-free supernatants were concentrated and freeze-dried to obtain fermented cassia seed powders.

TABLE 1

| Symbol | Strain | Acquisition method | Medium |
|---|---|---|---|
| L1 | Lactobacillus kefiri MJ90 | Isolated from Tibetan | MRS |
| | | mushroom | |
| L2 | Lactobacillus paracasei | Isolated from 2-year | MRS |
| | | ripened kimchi | |
| L3 | Lactobacillus acidophillus | Korea Food Research | MRS |
| | 128 | Institute | |
| L4 | Lactobacillus plantarum | Korea Food Research | MRS |
| | 144 | Institute | |
| B1 | Bifidobacterium longum | Korea Food Research | RCM/ |
| | | Institute | GAM |
| P1 | Pediococcus pentosaceus | Korea Food Research | MRS |
| | | Institute | |
| S1 | Saccharomyces cerevisiae | Commercially | YM |
| | | available yeast | |

### Experimental Example 1: Identification of Fermentation Characteristics of the Fermented Products of Cassia Seeds

The fermentation characteristics of the lactic acid fermented products and the yeast fermented product of cassia seeds prepared in Preparative Example 4 were identified by investigating changes in the pH of the fermented products and the number of viable bacteria at 36 and 72 hours after inoculation.

The number of viable bacteria was measured by the following procedure. First, 1 ml of each sample was diluted and sufficiently mixed with 9 ml of sterile physiological saline. The mixture was diluted stepwise and its portion (0.1 ml) was taken. The sample portion was plated on MRS agar for Lactobacillus and Pediococcus and BL agar containing 5% horse blood for Bifidobacterium. The bacteria were stored in anaerobic jars (BBL Gas Pak Anaerobic System) while maintaining an anaerobic environment if possible. After 24-48 h culture at 37□, the numbers of colonies formed were counted. The Saccharomyces strain was plated on YM agar and cultured at 25° C. for 48 h. The number of colonies formed was counted. The average numbers of colonies were multiplied by the dilution factors to calculate the numbers of colonies per culture solution.

A. PH Changes

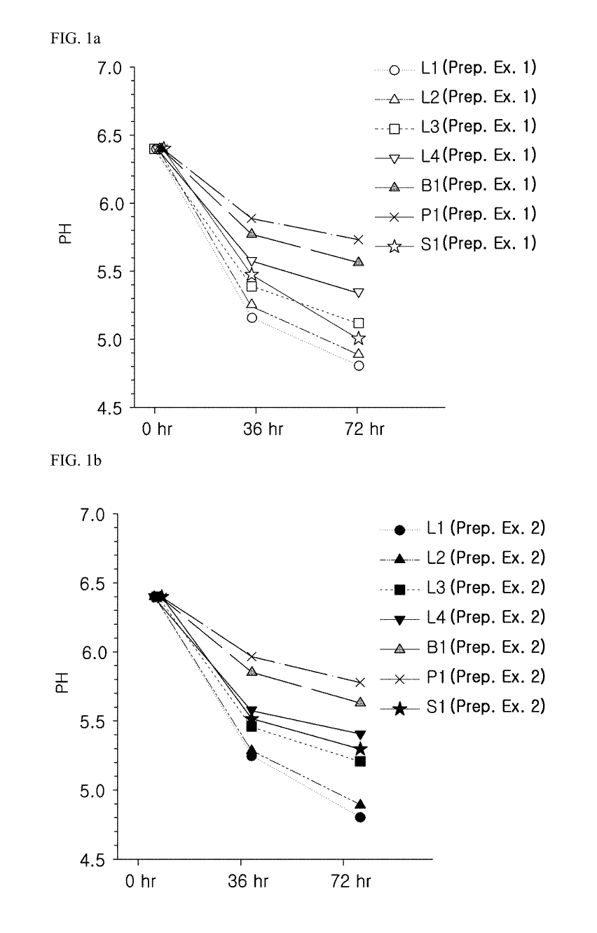

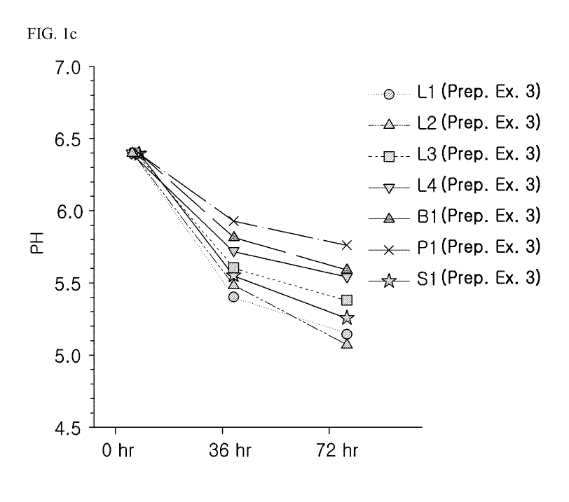

Changes in the pH of the fermented products of the extract of Preparative Example 1, the extract of Preparative Example 2, and the dilution of Preparative Example 3 depending on the kind of the fermentation bacteria and the culture time are shown in FIGS. 1a, 1b, and 1c, respectively.

The pH decreased from the initial value (6.4) to 5.2-5.8 at 36 h and 4.9-5.5 at 72 h.

The pH values decreased considerably for Lactobacillus kefiri MJ90 (L1) and Lactobacillus paracasei (L2) compared to those for Lactobacillus acidophilus 128 (L3), Lactobacillus plantarum 144 (L4), Bifidiobacterium longum (B1), and Pediococcus pentosaceus. In the extract of Preparative Example 1 and the dilution of Preparative Example 3, the pH values for Saccharomyces cerevisiae (S1) decreased to an intermediate level between those for L2 and L3. In the extract of Preparative Example 2, the pH values for Saccharomyces cerevisiae (S1) decreased to a level similar to those for L3.

For the same fermentation bacteria, there were almost no differences in the pH of the culture solutions of the hot water extract of Preparative Example 1, the ethanolic extract of Preparative Example 2, and the aqueous dilution of Preparation 3 as substrates. The pH values of the culture solutions of the aqueous dilution of Preparation 3 were slightly higher at 36 h and 72 h than those of the others.

B. Changes in the Number of Viable Bacteria

The six fermentation bacterial strains were precultured and their growth abilities were investigated. To this end, 1 wt % of each lactic acid bacterial strain was inoculated into the cassia seed extracts, each having a solid content of 6 wt %, followed by fermentation at 37° C. for 24 h. 1 wt % of the yeast strain was inoculated and fermented for 24 h. The numbers of viable bacteria were measured. The results are shown in Table 2.

TABLE 2

| | Number of viable bacteria (CFU/ml) | | | |
|---|---|---|---|---|
| | | After | After | After |
| | | fermentation | fermentation | fermentation |
| | Before | (Preparative | (Preparative | (Preparative |
| Symbol | fermentation | Example 1) | Example 2) | Example 3) |
| L1 | 5.0 × 10⁵ | 7.2 × 10⁸ | 6.9 × 10⁸ | 3.3 × 10⁸ |
| L2 | 5.0 × 10⁵ | 2.6 × 10⁸ | 2.2 × 10⁸ | 1.0 × 10⁸ |
| L3 | 5.0 × 10⁵ | 8.2 × 10⁷ | 8.1 × 10⁷ | 4.3 × 10⁷ |
| L4 | 5.0 × 10⁵ | 5.4 × 10⁷ | 6.3 × 10⁷ | 3.2 × 10⁷ |
| B1 | 5.0 × 10⁵ | 8.8 × 10⁶ | 1.2 × 10⁷ | 3.2 × 10⁶ |
| P1 | 5.0 × 10⁵ | 7.5 × 10⁶ | 8.1 × 10⁶ | 1.2 × 10⁶ |
| S1 | 5.0 × 10⁵ | 1.5 × 10⁸ | 1.2 × 10⁸ | 8.6 × 10⁷ |

As can be seen from the results in Table 2, there were no substantial differences in the number of viable bacteria when the hot water extract of Preparative Example 1 and the aqueous ethanolic extract of Preparative Example 2 were used as substrates. The numbers of viable bacteria after fermentation of the aqueous dilution of Preparative Example 3 was used were slightly smaller than half of those after fermentation of the extracts of Preparative Examples 1 and 2.

The numbers of viable bacteria in the culture solutions prepared using the Lactobacillus kefiri MJ90 (L1) strain were about 3 times larger than those in the culture solutions prepared using the Lactobacillus paracasei (L2) strain and were about 5 times larger than those in the culture solutions prepared using the Saccharomyces cerevisiae (S1).

The numbers of viable bacteria in the culture solutions prepared using the Lactobacillus acidophillus 128 (L3) strain and Lactobacillus plantarum 144 (L4) strain were about one-tenth of those in the culture solutions prepared using the L1 strain. The numbers of viable bacteria in the culture solutions prepared using Bifidiobacterium longum (B1) and Pediococcus pentosaceus (P1) were about one-hundredth of those in the culture solutions prepared using the L1 strain.

Therefore, the Lactobacillus kefiri MJ90 (L1), Lactobacillus paracasei (L2), and Saccharomyces cerevisiae (S1) strains were selected because of their excellent fermentation characteristics in terms of pH reduction and high viability. The Lactobacillus acidophilus 128 (L3) and Bifidiobacterium longum (B1) strains were selected as controls with poor fermentation characteristics. The fermented products were used to confirm whether they are effective in ameliorating or preventing constipation compared to the cassia seed extracts through animal experiments.

### Experimental Example 2: Identification of Effect of the Extracts and the Fermented Products on the Prevention of Constipation Through Animal Experiments According to the Kind of Fermentation Bacteria

A. Experimental Animals and Experimental Groups

Four-week-old male Sprague-Dawley (SD) rats were purchased from Damul Science (Daejeon, Korea) and bred at a temperature of 20±2° C. and a humidity of 55±5% on a 12 h light-dark cycle. The experimental animals were acclimated for 1 week after purchase and were divided into the experimental groups (5 animals per group) shown in Table 3.

TABLE 3

| Symbol | Experimental group |
|---|---|
| NOR | Normal control receiving normal feed |
| LOP | Negative control administered subcutaneously 4 mg/kg of |
| | loperamide for 5 days to induce constipation |
| CON1 | Positive control administered subcutaneously 4 mg/kg of |
| | loperamide for 5 days after oral administration of 50 |
| | mg/kg of sennoside for 1 week |
| CON2 | Positive control administered subcutaneously 4 mg/kg of |
| | loperamide for 5 days after oral administration of 36.25 |
| | mg/kg of Dulcolax-S for 1 week |
| 2-200 | Group administered subcutaneously 4 mg/kg of loperamide for |
| | 5 days after oral administration of 200 mg/kg of the cassia |
| | seed extract of Preparative Example 2 for 1 week |
| 2-L1-100 | Group administered subcutaneously 4 mg/kg of loperamide for |
| | 5 days after oral administration (1 week) of 100 mg/kg of |
| | the product of Preparative Example 4 obtained by |
| | fermentation of the extract of Preparative Example 2 with |
| | L1 strain |
| 2-L1-200 | Group administered subcutaneously 4 mg/kg of loperamide for |
| | 5 days after oral administration (1 week) of 200 mg/kg of |
| | the product of Preparative Example 4 obtained by |
| | fermentation of the extract of Preparative Example 2 with |
| | L1 strain |
| 2-L1-500 | Group administered subcutaneously 4 mg/kg of loperamide for |
| | 5 days after oral administration (1 week) of 500 mg/kg of |
| | the product of Preparative Example 4 obtained by |
| | fermentation of the extract of Preparative Example 2 with |
| | L1 strain |
| 2-L2-100 | Group administered subcutaneously 4 mg/kg of loperamide for |
| | 5 days after oral administration (1 week) of 100 mg/kg of |
| | the product of Preparative Example 4 obtained by |
| | fermentation of the extract of Preparative Example 2 with |
| | L2 strain |
| 2-L2-200 | Group administered subcutaneously 4 mg/kg of loperamide for |
| | 5 days after oral administration (1 week) of 200 mg/kg of |
| | the product of Preparative Example 4 obtained by |
| | fermentation of the extract of Preparative Example 2 with |
| | L2 strain |
| 2-L2-500 | Group administered subcutaneously 4 mg/kg of loperamide for |
| | 5 days after oral administration (1 week) of 500 mg/kg of |
| | the product of Preparative Example 4 obtained by |
| | fermentation of the extract of Preparative Example 2 with |
| | L2 strain |
| 2-L3-100 | Group administered subcutaneously 4 mg/kg of loperamide for |
| | 5 days after oral administration (1 week) of 100 mg/kg of |
| | the product of Preparative Example 4 obtained by |
| | fermentation of the extract of Preparative Example 2 with |
| | L3 strain |
| 2-L3-200 | Group administered subcutaneously 4 mg/kg of loperamide for |
| | 5 days after oral administration (1 week) of 200 mg/kg of |
| | the product of Preparative Example 4 obtained by |
| | fermentation of the extract of Preparative Example 2 with |
| | L3 strain |
| 2-L3-500 | Group administered subcutaneously 4 mg/kg of loperamide for |
| | 5 days after oral administration (1 week) of 500 mg/kg of |
| | the product of Preparative Example 4 obtained by |
| | fermentation of the extract of Preparative Example 2 with |
| | L3 strain |
| 2-B1-100 | Group administered subcutaneously 4 mg/kg of loperamide for |
| | 5 days after oral administration (1 week) of 100 mg/kg |
| | of the product of Preparative Example 4 obtained by |
| | fermentation of the extract of Preparative Example 2 with |
| | B1 strain |
| 2-B1-200 | Group administered subcutaneously 4 mg/kg of loperamide for |
| | 5 days after oral administration (1 week) of 200 mg/kg of |
| | the product of Preparative Example 4 obtained by |
| | fermentation of the extract of Preparative Example 2 with |
| | B1 strain |
| 2-B1-500 | Group administered subcutaneously 4 mg/kg of loperamide for |
| | 5 days after oral administration (1 week) of 500 mg/kg of |
| | the product of Preparative Example 4 obtained by |
| | fermentation of the extract of Preparative Example 2 with |
| | B1 strain |
| 2-S1-100 | Group administered subcutaneously 4 mg/kg of loperamide for |
| | 5 days after oral administration (1 week) of 100 mg/kg of |
| | the product of Preparative Example 4 obtained by |
| | fermentation of the extract of Preparative Example 2 with |
| | S1 strain |
| 2-S1-200 | Group administered subcutaneously 4 mg/kg of loperamide for |
| | 5 days after oral administration (1 week) of 200 mg/kg of |
| | the product of Preparative Example 4 obtained by |
| | fermentation of the extract of Preparative Example 2 with |
| | S1 strain |
| 2-S1-500 | Group administered subcutaneously 4 mg/kg of loperamide for |
| | 5 days after oral administration (1 week) of 500 mg/kg of |
| | the product of Preparative Example 4 obtained by |
| | fermentation of the extract of Preparative Example 2 with |
| | S1 strain |

All experimental groups other than the normal control (NOR) and the negative control (LOP) were administered orally the respective drugs (positive controls) or the respective fermented products of Preparative Example 4 for 5 days before loperamide administration.

The negative control (LOP), the positive controls (CON1, CON2), and all experimental groups other than the normal control (NOR) were administered subcutaneously loperamide for 5 days to induce constipation. The experimental animals were sacrificed on day 6 after initiation of the loperamide administration.

B. Weight, Feed Intake and Water Intake

The body weight changes (g), feed intakes (g), and water intakes (mL) of the experimental animals in all experimental groups were investigated from the initiation of loperamide administration until the experimental animals were sacrificed. As a result, there were no significant differences in feed intake and water intake. No significant differences in weight gain were found.

C. Number of Feces

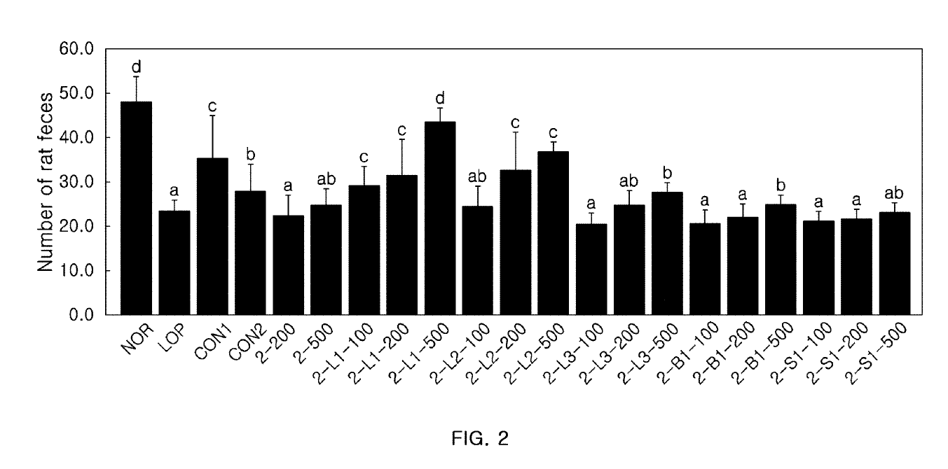

The numbers of feces from the experimental groups are shown in FIG. 2. 47.2±5.8 feces were observed in the normal control (NOR) whereas the number of feces was reduced to 23.1±3.6 in the loperamide-administered negative control (LOP), demonstrating that loperamide has the ability to induce constipation and sennoside (CON1) is more effective in increasing the number of feces than Dulcolax-S(CON2).

In the experimental group 2-200 administered 200 mg/kg of the aqueous ethanolic cassia seed extract of Preparative Example 2, a significant increase in the number of feces was not almost observed. The experimental groups 2-L1-200 and 2-L2-200 administered the same amount (200 mg/kg) as the experimental group 2-200 showed increased numbers of feces, which were equivalent to the sennoside-administered control (CON1).

The experimental group 2-500 administered a larger amount (500 mg/kg) of the aqueous ethanolic cassia seed extract of Preparative Example 2 still showed no significant increase in the number of feces. The experimental groups 2-L3-200 and 2-B1-200 administered 200 mg/kg showed no significant increases in the number of feces but the experimental groups 2-L3-500 and 2-B1-500 showed significant increases in the number of feces. However, no increase in the number of feces was found in the experimental group 2-S1-500 administered 500 mg/kg.

Taken together, the products obtained by fermentation with the Lactobacillus kefiri MJ90 (L1) strain and the Lactobacillus paracasei (L2) strain showed increases in the number of feces comparable or superior to the sennoside-administered group (CON1) and Dulcolax-S-administered group (CON2). The products obtained by fermentation with the Lactobacillus acidophillus 128 (L3) strain and the Bifidiobacterium longum (B1) strain showed increases in the number of feces only in the groups administered 500 mg/kg. No increases in the number of feces were observed in the groups fermented with the yeast strain Saccharomyces cerevisiae (S1).

These results concluded that the lactic acid bacteria fermented the cassia seed extracts to increase the number of feces irrespective of the different effective amounts of the fermented products. Particularly, the number of feces from the group 2-L1-500 administered 500 mg/kg of the product obtained by fermentation with Lactobacillus kefiri MJ90 (L1) strain increased to a level equivalent to that in the normal control (NOR), demonstrating that the group 2-L1-500 was most effective in increasing the number of feces.

D. Weight of Feces

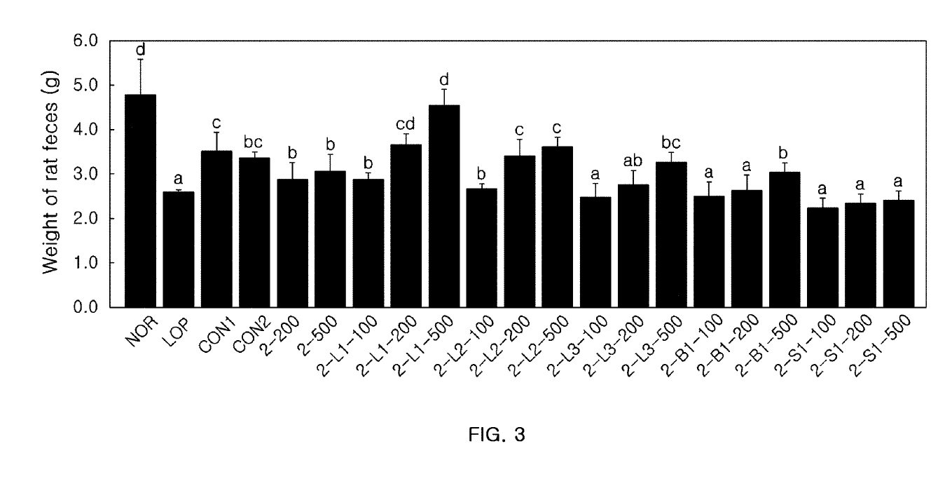

The weights of feces from the experimental groups were measured. The results are shown in FIG. 3.

The number of feces from the normal control (NOR) was 4.8±0.89 and that from the loperamide-administered negative control (LOP) was reduced to 2.6±0.32, demonstrating that loperamide has the ability to induce constipation and there was no significant difference in the weight of feces between the sennoside (CON1)-administered group and the Dulcolax-S-administered group (CON2).

The weight of feces from the experimental group 2-200 administered 200 mg/kg of the aqueous ethanolic cassia seed extract (Preparative Example 2) was smaller than that from the sennoside-administered group (CON1) but was significantly larger than that from the negative control (LOP). As in the experimental group 2-200, the experimental groups 2-L1-200 and 2-L2-200 administered 200 mg/kg showed increases in the weight of feces, which were comparable to the sennoside-administered group (CON1) and were superior to the Dulcolax-S-administered group (CON2).

The weight of feces from the experimental group 2-500 administered a larger amount of 500 mg/kg of the aqueous ethanolic cassia seed extract (Preparative Example 2) was similar to that from the experimental group 2-200, demonstrating that the increased amount of the aqueous ethanolic cassia seed extract did not lead to a notable increase in the weight of feces. In contrast, the experimental groups 2-L3-500 and 2-B1-500 showed significant increases in the weight of feces although the groups 2-L3-200 and 2-B1-200 administered 200 mg/kg showed no significant increases in the weight of feces. However, there was no increase in the weight of feces from the experimental group 2-S1-500 administered 500 mg/kg.

These results concluded that the product obtained by fermentation with the Lactobacillus kefiri MJ90 (L1) strain was more effective in increasing the weight of feces than sennoside (CON1) and the effect of the product obtained by fermentation with the Lactobacillus paracasei (L2) strain to increase the weight of feces was comparable or superior to that of the sennoside-administered group (CON1) and the Dulcolax-S-administered group. The product obtained by fermentation with the Lactobacillus acidophilus 128 (L3) strain and the product obtained by fermentation with the Bifidiobacterium longum (B1) strain were less effective in increasing the weight of feces than sennoside-administered group (CON1) and Dulcolax-S-administered group (CON2) but were effective in increasing the weight of feces in the groups administered 500 mg/kg. The product obtained by fermentation with the yeast strain Saccharomyces cerevisiae (S1) was not effective in increasing the weight of feces.

### Experimental Example 3: Amelioration and Prevention of Constipation Through Animal Experiments According to the Kind of Extracts

A. Experimental Animals and Experimental Groups

Experimental animals were prepared in the same manner as in Experimental Example 2. The experimental animals were acclimated for 1 week after purchase and were divided into the experimental groups (5 animals per group) shown in Table 4.

TABLE 4

| Symbol | Experimental group |
|---|---|
| NOR | Normal control receiving normal feed |
| LOP | Negative control administered subcutaneously 4 mg/kg of |
| | loperamide for 5 days to induce constipation |
| CON1 | Positive control administered subcutaneously 4 mg/kg of |
| | loperamide for 5 days after oral administration of 50 |
| | mg/kg of sennoside for 1 week |
| CON2 | Positive control administered subcutaneously 4 mg/kg of |
| | loperamide for 5 days after oral administration of 36.25 |
| | mg/kg of Dulcolax-S for 1 week |
| 1-200 | Group administered subcutaneously 4 mg/kg of loperamide |
| | for 5 days after oral administration of 200 mg/kg of the |
| | cassia seed extract of Preparative Example 1 for 1 week |
| 1-L1-100 | Group administered subcutaneously 4 mg/kg of loperamide |
| | for 5 days after oral administration (1 week) of 100 |
| | mg/kg of the product of Preparative Example 4 obtained |
| | by fermentation of the extract of Preparative Example 1 |
| | with L1 strain |
| 1-L1-200 | Group administered subcutaneously 4 mg/kg of loperamide |
| | for 5 days after oral administration (1 week) of 200 |
| | mg/kg of the product of Preparative Example 4 obtained |
| | by fermentation of the extract of Preparative Example 1 |
| | with L1 strain |
| 1-L1-500 | Group administered subcutaneously 4 mg/kg of loperamide |
| | for 5 days after oral administration (1 week) of 500 |
| | mg/kg of the product of Preparative Example 4 obtained |
| | by fermentation of the extract of Preparative Example 1 |
| | with L1 strain |
| 3-L1-100 | Group administered subcutaneously 4 mg/kg of loperamide |
| | for 5 days after oral administration (1 week) of 100 |
| | mg/kg of the product of Preparative Example 4 obtained |
| | by fermentation of the dilution of Preparative Example |
| | 3 with L1 strain |
| 3-L1-200 | Group administered subcutaneously 4 mg/kg of loperamide |
| | for 5 days after oral administration (1 week) of 200 |
| | mg/kg of the product of Preparative Example 4 obtained |
| | by fermentation of the dilution of Preparative Example |
| | 3 with L1 strain |
| 2-L1-200 (P) | Group administered orally 100 mg/kg of the product of |
| | Preparative Example 4 obtained by fermentation of the |
| | extract of Preparative Example 2 for 1 week after |
| | subcutaneous administration of 4 mg/kg of loperamide |
| | for 5 days |

1-L1-100 and 1-L1-200 were groups administered the fermented product of the aqueous cassia seed extract and 3-L1-100 and 3-L1-200 were groups administered the fermented product of the aqueous dilution. In 2-L1-200 (P), constipation was induced by pretreatment with loperamide to confirm the therapeutic effect of the fermented product on constipation.

All experimental groups other than the normal control (NOR), the negative control (LOP), and the group 2-L1-200 (P) were administered orally the respective drugs (positive controls) or the respective fermented products of Preparative Example 4 for 5 days before loperamide administration.

The negative control (LOP), the positive controls (CON1, CON2), and all experimental groups other than the normal control (NOR) were administered subcutaneously loperamide for 5 days to induce constipation. The experimental animals were sacrificed on day 6 after initiation of the loperamide administration.

In the group 2-L1-200 (P), loperamide was administered subcutaneously for 5 days to induce constipation in the same manner as in the negative control (LOP), and thereafter, 200 mg/kg of the product of Preparative Example 4 obtained by fermentation of the extract of Preparative Example 2 with the L1 strain was administered orally in the same manner as in the group 2-L1-200. The experimental animals were sacrificed on the next day after the last administration of the fermented product.

B. Weight, Feed Intake and Water Intake

The weight changes (g), feed intakes (g), and water intakes (mL) of the experimental animals in all experimental groups other than the group 2-L1-200 (P) were investigated from the initiation of loperamide administration until the experimental animals were sacrificed. The group 2-L1-200 (P) was measured for weight change (g), feed intake (g), and water intake (mL) from the initiation of the administration of the fermented product until the experimental animals were sacrificed. As a result, there were no significant differences in feed intake and water intake during the experimental period. No significant differences in weight gain were found.

C. Number of feces

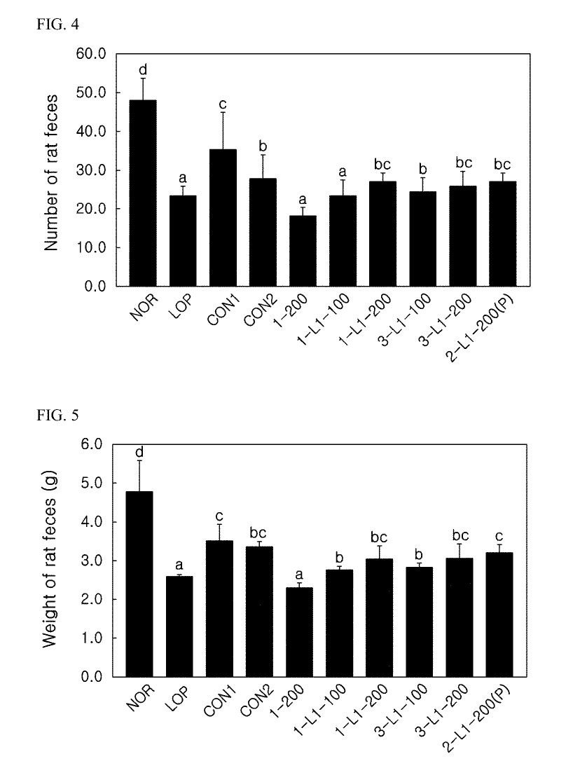

The numbers of feces from the experimental groups are shown in FIG. 4.

For the group 2-L1-200 (P), feces were removed from the cage at 4 days from the initiation of the administration of the fermented product. For the other groups, feces were removed from the cages at 4 days from the initiation of loperamide administration. After the beds were replaced with new ones, feces were collected for 1 day and the weights and numbers of the feces were measured.

The experimental groups 1-L1-200 and 3-L1-200 administered 200 mg/kg of the aqueous cassia seed extract of Preparative Example 1 showed significant increases in the number of feces compared to the experimental group 1-200 administered the same amount of the aqueous cassia seed extract. The experimental group 3-L1-100 administered a smaller amount (100 mg/kg) showed a significant increase in the number of feces, which was a level comparable to that of Dulcolax-S(CON2).

26.2±3.1 feces were observed in the experimental group 2-L1-200 (P) administered the product obtained by fermentation of the aqueous ethanolic cassia seed extract of Preparative Example 2 by fermentation with the L1 strain after the induction of constipation and the number of feces was significantly larger than that observed in the negative control (LOP).

D. Weight of Feces

The weights of feces from the experimental groups are shown in FIG. 5.

Compared to the experimental group 1-200 administered 200 mg/kg of the aqueous cassia seed extract of Preparative Example 1, the experimental groups 1-L1-200 and 3-L1-200 administered the same amount showed significant increases in the weight of feces. Each of the experimental groups 1-L1-100 and 3-L1-100 administered a smaller amount (100 mg/kg) also showed a significant increase in the weight of feces, which was a level comparable to that of Dulcolax-S(CON2).

3.18±0.28 feces were observed in the experimental group 2-L1-200 (P) and the number of feces was significantly larger than that observed in the negative control (LOP).

E. Colonic Transits of Carmine Dye

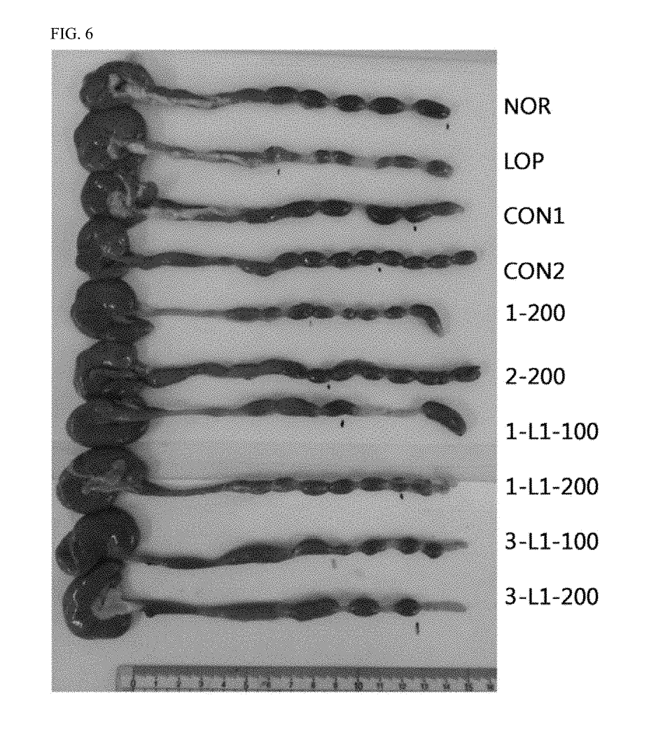

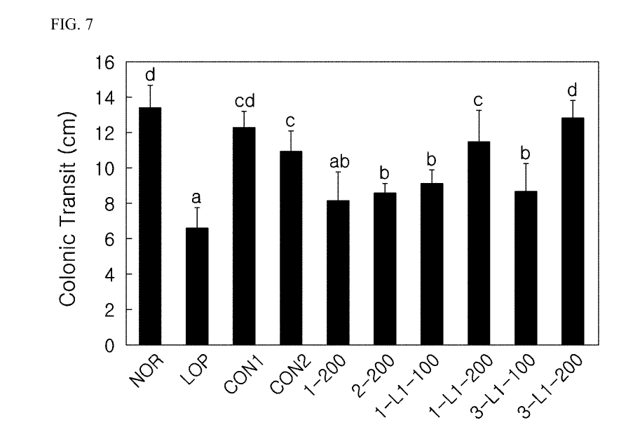

At day 6, 3 mL of carmine dye at a concentration of 1.5% (w/v) in physiological saline was administered orally to the experimental animals. 20 min after the oral administration, the experimental animals were sacrificed. Thereafter, in each experimental animal, both ends of the site of the colon from the appendix to the rectum were ligated. The colon site was excised and washed with PBS. The image of the washed colon site is shown in FIG. 6. The colonic transits of the carmine dye were measured from FIG. 6. The results are shown in FIG. 7.

The colonic transit of the carmine dye in the normal control (NOR) was 13.4±0.94 cm, whereas that in the negative control (LOP) was decreased to 6.6±0.75 cm, demonstrating that constipation was induced by loperamide. The colonic transits of the carmine dye in the sennoside-administered group (CON1) and the Dulcolax-S-administered group (CON2) were significantly increased compared to that in the negative control (LOP).

There was no significant difference in the colonic transit of the carmine dye between the experimental group 1-200 and the negative control (LOP) but the colonic transit of the carmine dye in the experimental group 2-200 was significantly increased compared to that in the negative control (LOP).

The colonic transits of the carmine dye in the experimental groups 1-L1-100 and 3-L1-100 were similar to that in the experimental group 2-200 although the amount of the fermented product administered to the experimental groups 1-L1-100 and 3-L1-100 was a half of that of the extract administered to the experimental group 2-200.

The colonic transits of the carmine dye in the experimental groups 1-L1-200 and 3-L1-200, which were administered the same amount of the fermented product as the experimental groups 1-200 and 2-200, were much larger than that in the experimental group 2-200.

The colonic transits of the carmine dye in the experimental groups 1-L1-200 and 3-L1-200 were similar to or larger than those in the sennoside-administered positive control and the Dulcolax-S-administered positive control. Particularly, the colonic transit of the carmine dye in the experimental group 3-L1-200 was similar to that in the normal control (NOR).

### Experimental Example 4: Comparison of HPLC-IT/TOF MS Patterns Before and after Fermentation

IT/TOF MS (HPLC-IT/TOF MS system, Shimadzu, Japan) was conducted on the aqueous ethanolic cassia seed extract powder of Preparative Example 2 and the product of Preparative Example 4 obtained by fermentation of the aqueous ethanolic cassia seed extract powder of Preparative Example 2 with the L1 strain. The extract powder and the fermented product were used at the same concentration. The HPLC-IT/TOF MS patterns were compared. The patterns were measured on an Acquity UPLC C-18 column (1.7 m 2.1×100 mm) at a flow rate of 0.3 mL/min with a gradient from 10% aqueous acetonitrile solution to 70% aqueous acetonitrile solution.

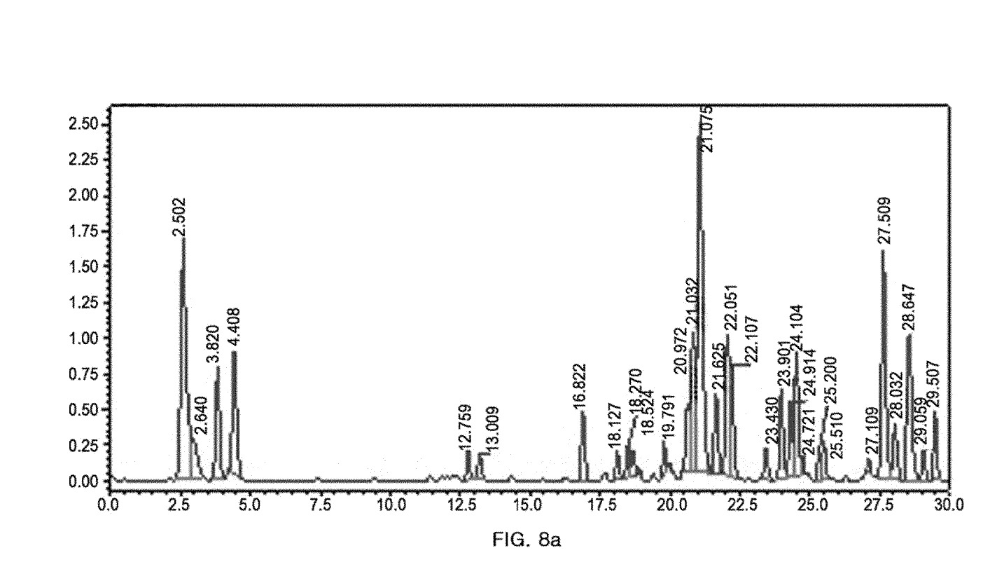

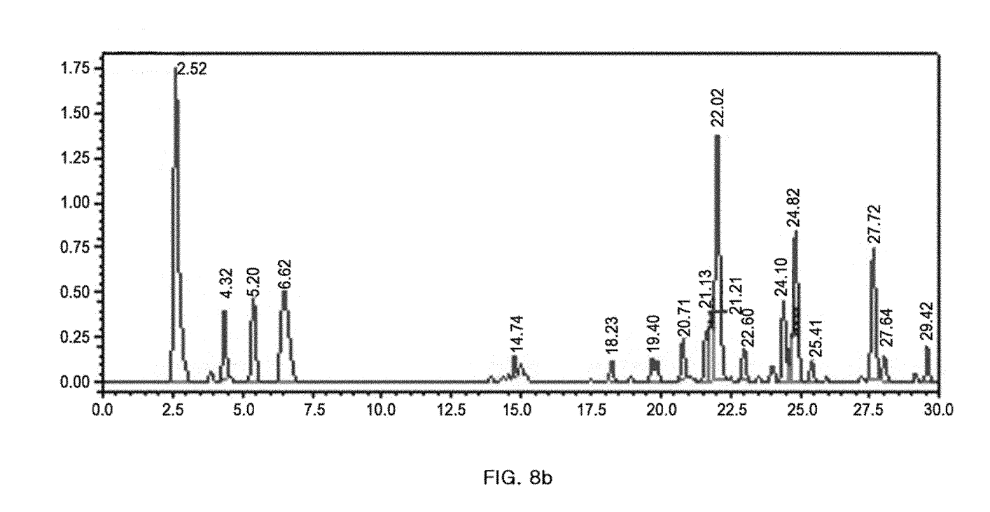

The patterns of the aqueous ethanolic cassia seed extract of Preparative Example 2 and the lactic acid fermented product of Preparative Example 3 are shown in FIGS. 8a and 8b, respectively. The pattern comparison revealed that many peaks disappeared or their intensities were reduced in the pattern of the aqueous ethanolic cassia seed extract (Preparative Example 2) and instead new peaks appeared after fermentation.

### Experimental Example 5: Identification of Lactobacillus kefiri MJ90 (L1) Strain

A. Strain Isolation

In the previous examples, the fermented product of cassia seeds prepared using Lactobacillus kefiri MJ90 was found to show superior activity in the amelioration or prevention of constipation. The strain was isolated by the following procedure.

The surface of one piece of Tibetan mushroom was washed several times with sterile water. The Tibetan mushroom piece was cultured with shaking at 37-150 rpm in MRS liquid medium for 24 h. The culture was diluted three times (each 10-fold) with sterile water in MRS plate medium containing 1% CaCO₃. 100 L of the culture sample diluted to 1×10³ times was plated onto MRS solid medium and cultured in an incubator under constant temperature and humidity conditions for 36-48 h. Morphologically different colonies were selected and cultured in MRS agar plate by streak plate method. The strain was isolated in a pure form.

B. Identification of the Strain Through 16s-rDNA Sequencing

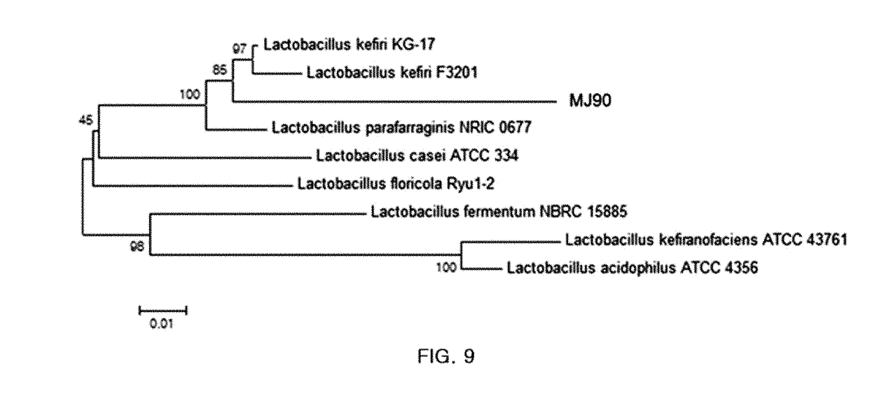

For sequencing, the isolated Lactobacillus kefiri MJ90 strain was cultured in Lactobacilli MRS broth, and 1.5 mL of the culture solution was centrifuged and washed with 0.8% sterile physiological saline. Chromosomal DNA was extracted using a genomic DNA kit and used as a template DNA for PCR. 9F (5′-GAG TTT GAT CCT GGC TCA G-3′) and 1412R (5′-GAG TTT G-3′) primers were used to amplify 16s rRNA genes from the bacteria. Amplified products were confirmed by electrophoresis of the PCR products and purified using a QIAquick PCR purification kit (QIAGEN, Hilden, Germany). The nucleotide sequence was determined using ABI PRISM Big Dye™ Terminator Cycle Sequencing Kits (Applied Biosystems, USA) and ABI PRISM 3730xl Analyzer (Applied Biosystems) in Geno Tech Co. (Daejeon, Korea). Blast searching was performed using the provided nucleotide sequence at the NCBI. The lineage was examined using the MEGA 5.0 program (FIG. 9) and the strain was identified as Lactobacillus capryi.

The 16s rRNA nucleotide sequence of Lactobacillus kefiri MJ90 is set forth in SEQ ID NO: 1. The strain was deposited at the Korean Culture Center of Microorganisms on Oct. 1, 2014 and received the accession number of KCCM11575P.

[Accession number]

Depositary authority: Korean Culture Center of Microorganisms (oversea)

Accession No.: KCCM11575P

Accession Date: 20141001

1. A health functional food for ameliorating or preventing constipation comprising, as an active ingredient, a lactic acid fermented product of cassia seeds.

2. The health functional food according to claim 1, wherein the cassia seeds are extracted with water, a C₁-C₄ alcohol or a mixed solvent thereof.

3. The health functional food according to claim 2, wherein the health functional food is in the form of a capsule, tablet, powder, granule, liquid, pill, flake, paste, syrup, gel, jelly or bar.

4. A pharmaceutical composition for treating or preventing constipation comprising, as an active ingredient, a lactic acid fermented product of cassia seeds.

5. The pharmaceutical composition according to claim 4, wherein the cassia seeds are extracted with water, a C₁-C₄ alcohol or a mixed solvent thereof.

6. A method for preparing a lactic acid fermented product of cassia seeds, comprising: preparing a cassia seed extract; and inoculating lactic acid bacteria into the cassia seed extract, followed by fermentation wherein the lactic acid fermented product increases the number, water content, and/or weight of feces.

7. The method according to claim 6, wherein the lactic acid bacteria are selected from the group consisting of lactic acid bacteria belonging to the genera Lactobacillus, Bifidobacterium, Leuconostoc, Pediococcus, and Enterococcus.

8. The method according to claim 7, wherein the lactic acid bacterial strain is Lactobacillus kefiri or Lactobacillus acidophilus.

9. The method according to claim 8, wherein the lactic acid bacterial strain is Lactobacillus kefiri MJ90 [Deposit number: KCCM 11575P].

10. The method according to claim 6, wherein the cassia seeds or their powder is extracted with water, a C₁-C₄ alcohol or a mixed solvent thereof; and the cassia seed extract is inoculated with 1×10³ to 1×10⁶ CFU/ml of lactic acid bacteria and fermented at 30 to 45° C. for 1 to 7 days.

11. The method according to claim 10, wherein the extraction is performed by mixing 1 part by weight of the cassia seed powder with 10 to 50 parts by weight of water and allowing the mixture to stand or stirring the mixture for 0.5 to 24 hours.

12. The method according to claim 10, wherein the extraction is performed by extracting 1 part by weight of the cassia seeds or their powder with 20 to 80% by weight of an aqueous ethanolic solution for 0.5 to 24 hours and drying the extract to remove the ethanol.

13. The method according to claim 10, wherein the cassia seed extract is fermented in the presence of one or more nutrients for the lactic acid bacteria.

14. Lactobacillus kefiri MJ90 (deposit number: KCCM11575P) that ferments cassia seeds to enhance the activity of the cassia seeds in the amelioration or prevention of constipation.

15. A method for treating or preventing constipation, comprising administering to a patient in need of such treatment or prevention an effective amount of a lactic acid fermented product of cassia seeds.
